(19) United States
(12) Patent Application Publication
(10) Pub. No.: US 2024/0070544 A1
(43) Pub. Date: Feb. 29, 2024

(54) MODEL GENERATION APPARATUS, DOCUMENT GENERATION APPARATUS, MODEL GENERATION METHOD, DOCUMENT GENERATION METHOD, AND PROGRAM

(71) Applicant: FUJIFILM Corporation, Tokyo (JP)

(72) Inventors: Shotaro MISAWA, Tokyo (JP); Hirokazu YARIMIZU, Tokyo (JP); Ryuji KANO, Tokyo (JP); Tomoki TANIGUCHI, Tokyo (JP); Tomoko OHKUMA, Tokyo (JP); Kohei ONODA, Tokyo (JP)

(73) Assignee: FUJIFILM Corporation, Tokyo (JP)

(21) Appl. No.: 18/458,130

(22) Filed: Aug. 29, 2023

(30) Foreign Application Priority Data
Aug. 31, 2022 (JP) 2022-138806

(51) Int. Cl. G06N 20/00; G16H 10/60

(52) U.S. Cl. CPC G06N 20/00; G16H 10/60

(57) ABSTRACT

A model generation apparatus includes a processor that is configured to: acquire information data for learning, acquire document data for learning, extract a first portion and a second portion having a lower rate of match with the information data than the first portion from the document data based on a rate of match between the information data and each portion of the document data, generate a first machine learning model by using first learning data in which first data for learning included in the information data is used as input data and the first portion is used as correct answer data, and generate a second machine learning model by using second learning data in which second data for learning included in the information data is used as input data and the second portion is used as correct answer data.

### CROSS-REFERENCE TO RELATED APPLICATION

This application claims priority from Japanese Patent Application No. 2022-138806, filed Aug. 31, 2022, the disclosure of which is incorporated herein by reference in its entirety.

### BACKGROUND

### Technical Field

The present disclosure relates to a model generation apparatus, a document generation apparatus, a model generation method, a document generation method, and a non-transitory storage medium storing a program.

### Related Art

The technology of generating, from information data representing information up to now, a document having a low degree of association to the information data is known. Examples of the document having a low degree of association include a document related to the future. For example, JP2020-119383A discloses the technology of generating transplantation candidate information including a result of a transplantation prognosis of a first patient by applying application data generated by using patient information related to the first patient and information related to a donor acquired from a database to a learning device that learns a relationship between the patient information, the information related to the donor, information related to a relationship between the patient and the donor, and the result of the transplantation prognosis.

By the way, in a case in which a document having a high degree of association to the information data and the document having a low degree of association to the information data are generated from the information data, an appropriate document may not be generated. For example, in the technology disclosed in JP2020-119383A, in a case in which the application data and the result of the transplantation prognosis are generated by using the learning device, at least one of the generated application data or the generated result of the transplantation prognosis may not be an appropriate document.

### SUMMARY

The present disclosure has been made in view of the above circumstances, and is to provide a model generation apparatus, a document generation apparatus, a model generation method, a document generation method, and a non-transitory storage medium storing a program capable of appropriately generating a document having a high degree of association to information data and a document having a low degree of association to the information data from the information data.

In order to achieve the object described above, a first aspect of the present disclosure relates to a model generation apparatus comprising at least one processor, in which the processor acquires information data for learning, acquires document data for learning, extracts a first portion and a second portion having a lower rate of match with the information data for learning than the first portion from the document data for learning based on a rate of match between the information data for learning and each portion of the document data for learning, generates a first machine learning model by using first learning data in which first data for learning included in the information data for learning is used as input data and the first portion is used as correct answer data, and generates a second machine learning model by using second learning data in which second data for learning included in the information data for learning is used as input data and the second portion is used as correct answer data, which is also called as gold data or target document.

A second aspect relates to the model generation apparatus according to the first aspect, in which the document data for learning is peculiar data related to a specific subject, which is any one of a specific individual, a specific object, or a specific event, with which first date information is associated, the information data for learning includes a plurality of document data which are peculiar data related to the specific subject with which the first date information or second date information indicating a date earlier than a date indicated by the first date information is associated.

A third aspect relates to the model generation apparatus according to the first aspect, in which the processor generates a third machine learning model that uses the document data for learning as input, and outputs at least one of the first portion or the second portion through reinforcement learning in which performance of the first machine learning model and performance of the second machine learning model are used as rewards, and extracts the first portion and the second portion from the document data for learning by using the third machine learning model.

A fourth aspect relates to the model generation apparatus according to the first aspect, in which the second machine learning model is a machine learning model that includes a machine learning model outputting a prediction result based on the information data for learning, and outputs a combination of the prediction result and a template.

In addition, in order to achieve the object described above, a fifth aspect of the present disclosure relates to a document generation apparatus comprising a first machine learning model generated by using first learning data in which first data for learning included in information data for learning is used as input data and a first portion extracted from document data for learning based on a rate of match between the information data for learning and each portion of the document data for learning is used as correct answer data, a second machine learning model generated by using second learning data in which second data for learning included in the information data for learning is used as input data and a second portion, which is extracted from the document data for learning and has a lower rate of match with the information data for learning than the first portion, is used as correct answer data, and at least one processor, in which the processor acquires information data, acquires a first document by inputting first data included in the information data to the first machine learning model, acquires a second document by inputting second data included in the information data to the second machine learning model, and generates a third document from the first document and the second document.

In addition, in order to achieve the object described above, a sixth aspect of the present disclosure relates to a model generation method executed by a processor of a model generation apparatus including at least one processor, the model generation method comprising acquiring information data for learning, acquiring document data for learning, extracting a first portion and a second portion having a lower rate of match with the information data for learning than the first portion from the document data for learning based on a rate of match between the information data for learning and each portion of the document data for learning, generating a first machine learning model by using first learning data in which first data for learning included in the information data for learning is used as input data and the first portion is used as correct answer data, and generating a second machine learning model by using second learning data in which second data for learning included in the information data for learning is used as input data and the second portion is used as correct answer data.

In addition, in order to achieve the object described above, a seventh aspect of the present disclosure relates to a document generation method executed by a processor of a document generation apparatus including a first machine learning model generated by using first learning data in which first data for learning included in information data for learning is used as input data and a first portion extracted from document data for learning based on a rate of match between the information data for learning and each portion of the document data for learning is used as correct answer data, a second machine learning model generated by using second learning data in which second data for learning included in the information data for learning is used as input data and a second portion, which is extracted from the document data for learning and has a lower rate of match with the information data for learning than the first portion, is used as correct answer data, and at least one processor, the document generation method comprising acquiring information data, acquiring a first document by inputting first data included in the information data to the first machine learning model, acquiring a second document by inputting second data included in the information data to the second machine learning model, and generating a third document from the first document and the second document.

In addition, in order to achieve the object described above, an eighth aspect of the present disclosure relates to a program for executing at least one of the model generation method according to the present disclosure or the document generation method according to the present disclosure.

According to the present disclosure, the document having a high degree of association to the information data and the document having a low degree of association to the information data can be appropriately generated from the information data.

### BRIEF DESCRIPTION OF THE DRAWINGS

FIG. 1 is a configuration diagram schematically showing an example of an overall configuration of a medical care summary generation system according to an embodiment.

FIG. 2 is a diagram for describing generation of a medical care summary by a medical care summary generation apparatus.

FIG. 3 is a block diagram showing an example of a configuration of the medical care summary generation apparatus.

FIG. 4 is a functional block diagram showing an example of a function related to the generation of the medical care summary of the medical care summary generation apparatus according to the embodiment.

FIG. 5 is a flowchart showing an example of a flow of a medical care summary generation process by the medical care summary generation apparatus according to the embodiment.

FIG. 6 is a diagram showing an example of a state in which a relation document and the medical care summary are displayed on a display unit.

FIG. 7 is a functional block diagram showing an example of the function related to the generation of the medical care summary of the medical care summary generation apparatus according to the embodiment.

FIG. 8 is a diagram for describing an action of a past sentence and future sentence definition mechanism.

FIG. 9 is a diagram for describing training a past sentence generation mechanism model by a past sentence summary generation mechanism learning unit.

FIG. 10 is a diagram for describing training a future sentence generation mechanism model by a future sentence summary generation mechanism learning unit.

FIG. 11 is a flowchart showing an example of a flow of a learning process by the medical care summary generation apparatus according to the embodiment.

FIG. 12 is a diagram for describing a past sentence and future sentence definition mechanism of a modification example 1.

### DESCRIPTION OF THE PREFERRED EMBODIMENTS

An embodiment of the present disclosure will be described below in detail with reference to the drawings. It should be noted that the present embodiment does not limit the technology of the present disclosure.

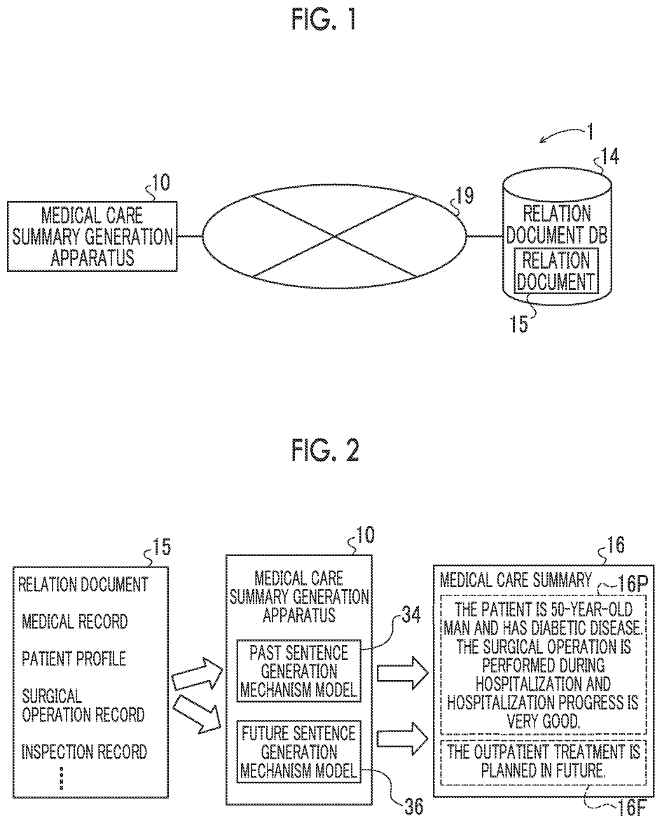

First, an example of an overall configuration of a medical care summary generation system according to the present embodiment will be described. FIG. 1 shows a configuration diagram showing an example of an overall configuration of a medical care summary generation system 1 according to the present embodiment. As shown in FIG. 1, the medical care summary generation system 1 according to the present embodiment comprises a medical care summary generation apparatus 10 and a relation document database (DB) 14. The medical care summary generation apparatus 10 and the relation document DB 14 that stores a relation document 15 related to each of a plurality of patients are connected via a network 19 by wired communication or wireless communication.

The relation document DB 14 stores the relation documents 15 related to the plurality of patients. The relation document DB 14 is realized by a storage medium, such as a hard disk drive (HDD), a solid state drive (SSD), and a flash memory, provided in a server apparatus in which a software program for providing functions of a database management system (DBMS) to a general-purpose computer is installed.

As an example, the relation document 15 according to the present embodiment is a document related to a medical care of the patient, and examples of the relation document 15 include, as patient information (or patient data), at least one of a medical record of the patient related to the patient, a patient profile, a surgical operation record, or an inspection record, as shown in FIG. 2. It should be noted that, in the present embodiment, the “document” is information in which at least one of a word or a sentence is used as a component. For example, the document may include only one word, or may include a plurality of sentences. The relation document 15 is stored in the relation document DB 14 in association with identification information for identifying the patient for each specific patient. The relation document 15 according to the present embodiment is an example of information data according to the present disclosure.

On the other hand, as shown in FIG. 2, the medical care summary generation apparatus 10 is an apparatus that includes a past sentence generation mechanism model 34 and a future sentence generation mechanism model 36, and generates a medical care summary 16 from the relation document 15 related to the specific patient. The medical care summary 16 is a medical care summary related to a medical care of the specific patient, and includes a past sentence summary 16P and a future sentence summary 16F.

The medical care summary generation apparatus 10 generates the past sentence summary 16P from the relation document 15 by using the past sentence generation mechanism model 34. The past sentence summary 16P is a medical care summary related to a medical care of the specific patient in the past from a point in time at which the medical care summary 16 is generated. So to speak, the past sentence summary 16P can be said to be a document summarizing the contents of the relation document 15, and is a document having a high rate of match with the relation document 15. As an example, two sentences, “The patient is a 50-year-old man and has a diabetic disease.” and “The surgical operation is performed during hospitalization and the hospitalization progress is very good.”, are included in the past sentence summary 16P shown in FIG. 2.

On the other hand, the medical care summary generation apparatus 10 generates the future sentence summary 16F from the relation document 15 by using the future sentence generation mechanism model 36. The future sentence summary 16F is a medical care summary related to a medical care of the specific patient in the future from a point in time at which the medical care summary 16 is generated, and is, for example, a medical care summary related to a prognosis prediction or a medical care plan of the specific patient. The future sentence summary 16F is a document having a low rate of match with the relation document 15. As an example, one sentence, “The outpatient treatment is planned in the future.”, is included in the future sentence summary 16F shown in FIG. 2. The medical care summary generation apparatus 10 according to the present embodiment is an example of a document generation apparatus according to the present disclosure.

In addition, the medical care summary generation apparatus 10 according to the present embodiment has a function of generating each of the past sentence generation mechanism model 34 used for the generation of the past sentence summary 16P and the future sentence generation mechanism model 36 used for the generation of the future sentence summary 16F. The medical care summary generation apparatus 10 according to the present embodiment is an example of a model generation apparatus according to the present disclosure. It should be noted that the details of the generation of the past sentence generation mechanism model 34 and the future sentence generation mechanism model 36 will be described below.

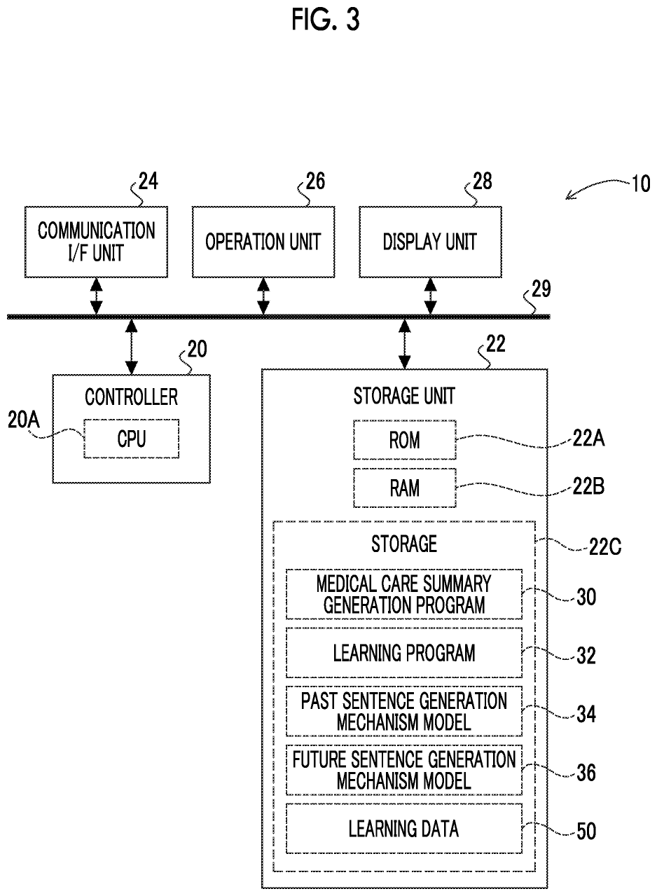

As shown in FIG. 3, the medical care summary generation apparatus 10 according to the present embodiment comprises a controller 20, a storage unit 22, a communication interface (UF) unit 24, an operation unit 26, and a display unit 28. The controller 20, the storage unit 22, the communication OF unit 24, the operation unit 26, and the display unit 28 are connected to each other via a bus 29, such as a system bus or a control bus, such that various types of information can be exchanged.

The controller 20 according to the present embodiment controls an overall operation of the medical care summary generation apparatus 10. The controller 20 is a processor, and comprises a central processing unit (CPU) 20A. Also, the controller 20 is connected to the storage unit 22 described below.

The operation unit 26 is used by a user to input, for example, an instruction or various types of information related to the generation of the medical care summary 16. The operation unit 26 is not particularly limited, and examples of the operation unit 26 include various switches, a touch panel, a touch pen, and a mouse. The display unit 28 displays the medical care summary 16, the relation document 15, various types of information, and the like. It should be noted that the operation unit 26 and the display unit 28 may be integrated to form a touch panel display.

The communication I/F unit 24 performs communication of various types of information with the relation document DB 14 via the network 19 by wireless communication or wired communication. The medical care summary generation apparatus 10 receives the relation document 15 related to the specific patient from the medical care summary 16 via the communication I/F unit 24 by wireless communication or wired communication.

The storage unit 22 comprises a read only memory (ROM) 22A, a random access memory (RAM) 22B, and a storage 22C. Various programs or the like executed by the CPU 20A are stored in advance in the ROM 22A. Various data are transitorily stored in the RAM 22B. The storage 22C stores a medical care summary generation program 30 and a learning program 32 executed by the CPU 20A. In addition, the storage 22C stores the past sentence generation mechanism model 34, the future sentence generation mechanism model 36, learning data 50, and various other information. The storage 22C is a non-volatile storage unit, and examples of the storage 22C include an HDD and an SSD.

Generation of Medical Care Summary 16

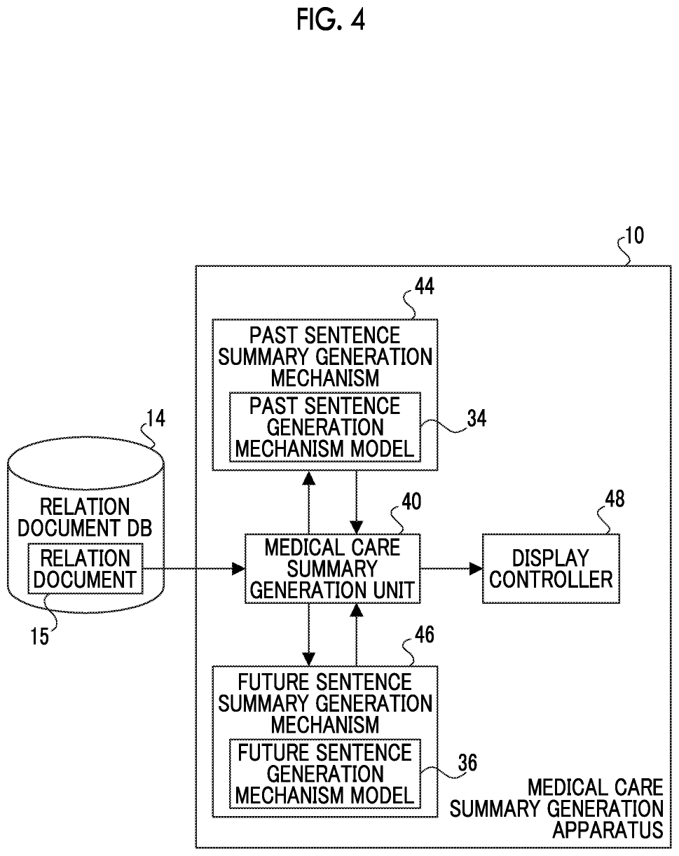

First, a function of generating the medical care summary 16 in the medical care summary generation apparatus 10 according to the present embodiment will be described. Stated another way, an operation phase of the past sentence generation mechanism model 34 and the future sentence generation mechanism model 36 will be described. FIG. 4 shows a functional block diagram of an example of a configuration related to the generation of the medical care summary 16 in the medical care summary generation apparatus 10 according to the present embodiment. As shown in FIG. 4, the medical care summary generation apparatus 10 comprises a medical care summary generation unit 40, a past sentence summary generation mechanism 44, a future sentence summary generation mechanism 46, and a display controller 48. As an example, in the medical care summary generation apparatus 10 according to the present embodiment, in a case in which the CPU 20A of the controller 20 executes the medical care summary generation program 30 stored in the storage 22C, the CPU 20A functions as the medical care summary generation unit 40, the past sentence summary generation mechanism 44, the future sentence summary generation mechanism 46, and the display controller 48.

In a case in which patient identification information indicating the specific patient for which the medical care summary is generated is received, the medical care summary generation unit 40 acquires the relation document 15 corresponding to the received patient identification information from the relation document DB 14 via the network 19. The medical care summary generation unit 40 outputs the acquired relation document 15 to the past sentence summary generation mechanism 44 and the future sentence summary generation mechanism 46.

In addition, the medical care summary generation unit 40 acquires the past sentence summary 16P generated by the past sentence summary generation mechanism 44 and the future sentence summary 16F generated by the future sentence summary generation mechanism 46, and generates the medical care summary 16 from the past sentence summary 16P and the future sentence summary 16F. The medical care summary generation unit 40 according to the present embodiment generates the medical care summary 16 from the past sentence summary 16P and the future sentence summary 16F based on a predetermined format. As an example, the medical care summary generation unit 40 according to the present embodiment generates the medical care summary 16 by adding the future sentence summary 16F after the past sentence summary 16P.

The past sentence summary generation mechanism 44 includes the past sentence generation mechanism model 34, and generates the past sentence summary 16P related to the specific patient from the relation document 15 by using the past sentence generation mechanism model 34. As an example, the past sentence summary generation mechanism 44 according to the present embodiment vectorizes the relation document 15 for each document or for each word included in the relation document 15 to input the vectorized relation document 15 to the past sentence generation mechanism model 34, and acquires the output past sentence summary 16P. The past sentence summary generation mechanism 44 outputs the generated past sentence summary 16P to the medical care summary generation unit 40. The past sentence generation mechanism model 34 according to the present embodiment is an example of a first machine learning model according to the present disclosure. In addition, the relation document 15 related to the specific patient according to the present embodiment is an example of first data according to the present disclosure. In addition, the past sentence summary 16P according to the present embodiment is an example of a first document according to the present disclosure.

The future sentence summary generation mechanism 46 includes the future sentence generation mechanism model 36, and generates the future sentence summary 16F related to the specific patient from the relation document 15 by using the future sentence generation mechanism model 36. As an example, the future sentence summary generation mechanism 46 according to the present embodiment vectorizes the relation document 15 for each document or for each word included in the relation document 15 to input the vectorized relation document 15 to the future sentence summary generation mechanism 46, and acquires the output future sentence summary 16F. The future sentence summary generation mechanism 46 outputs the generated future sentence summary 16F to the medical care summary generation unit 40. The future sentence generation mechanism model 36 according to the present embodiment is an example of a second machine learning model according to the present disclosure. In addition, the relation document 15 related to the specific patient according to the present embodiment is an example of second data according to the present disclosure. In addition, the future sentence summary 16F according to the present embodiment is an example of second document according to the present disclosure.

The display controller 48 performs control of displaying the medical care summary 16 generated by the medical care summary generation unit 40 on the display unit 28. In addition, the display controller 48 also performs control of displaying the relation document 15 of the specific patient, which is a source for the generation of the medical care summary 16, on the display unit 28.

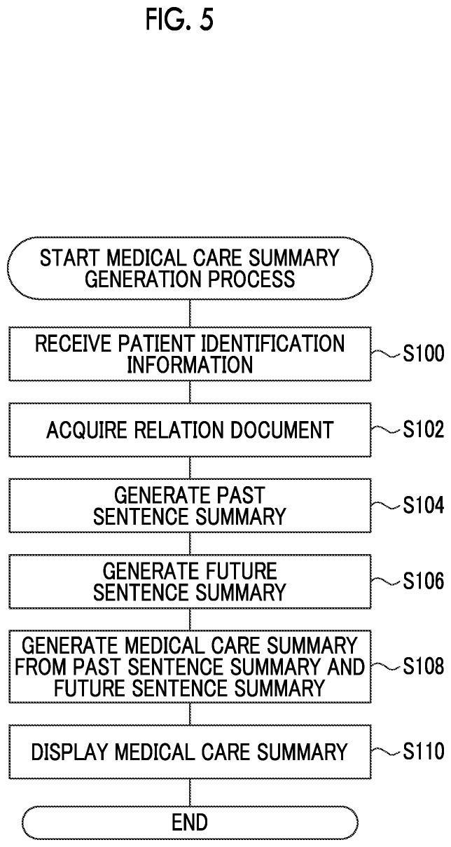

Next, an action of the generation of the medical care summary in the medical care summary generation apparatus 10 according to the present embodiment will be described with reference to the drawings. FIG. 5 shows a flowchart showing an example of a flow of a medical care summary generation process executed in the medical care summary generation apparatus 10 according to the present embodiment. As an example, the medical care summary generation apparatus 10 according to the present embodiment executes the medical care summary generation process shown in FIG. 5 as an example by the CPU 20A of the controller 20 executing the medical care summary generation program 30 stored in the storage 22C based on, for example, a start instruction performed by the user using the operation unit 26.

In step S100 of FIG. 5, the medical care summary generation unit 40 receives the patient identification information of the specific patient designated by the user using the operation unit 26, as described above. In next step S102, as described above, the medical care summary generation unit 40 acquires the relation document 15 associated with the patient identification information from the relation document DB 14 via the network 19. The acquired relation document 15 is output to the past sentence summary generation mechanism 44 and the future sentence summary generation mechanism 46.

In next step S104, as described above, the past sentence summary generation mechanism 44 generates the past sentence summary 16P by vectorizing the relation document 15 to input the vectorized relation document 15 to the past sentence generation mechanism model 34, and acquiring the output past sentence summary 16P. The past sentence summary generation mechanism 44 outputs the generated past sentence summary 16P to the medical care summary generation unit 40.

In next step S106, as described above, the future sentence summary generation mechanism 46 generates the future sentence summary 16F by vectorizing the relation document 15 to input the vectorized relation document 15 to the future sentence generation mechanism model 36, and acquiring the output future sentence summary 16F. The future sentence summary generation mechanism 46 outputs the generated future sentence summary 16F to the medical care summary generation unit 40.

It should be noted that an order in which steps S104 and S106 are executed is not particularly limited. For example, the process of step S106 may be executed before the process of step S104. Also, for example, the process of step S104 and the process of step S106 may be executed in parallel.

In next step S108, as described above, the medical care summary generation unit 40 generates the medical care summary 16 from the past sentence summary 16P generated by the past sentence summary generation mechanism 44 and the future sentence summary 16F generated by the future sentence summary generation mechanism 46.

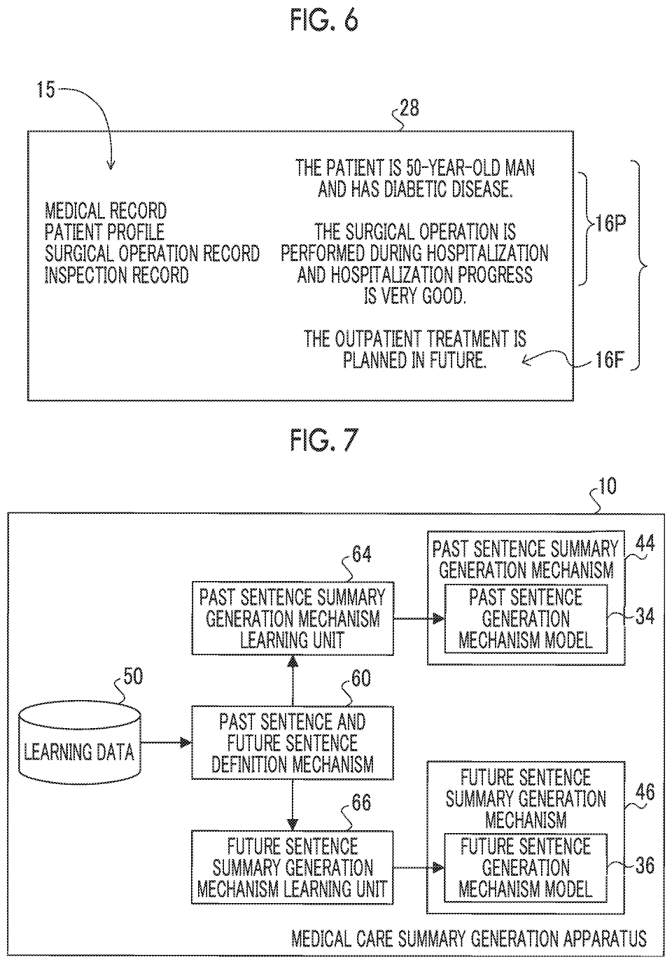

In next step S110, the display controller 48 displays the relation document 15 and the medical care summary 16 on the display unit 28 as described above. FIG. 6 shows an example of a state in which the relation document 15 and the medical care summary 16 are displayed on the display unit 28. As a result, the user, such as a doctor, can obtain the medical care summary 16 related to the specific patient. In a case in which the process of step S110 is terminated, the medical care summary generation process shown in FIG. 5 is terminated.

As described above, with the medical care summary generation apparatus 10 according to the present embodiment, the medical care summary 16 including the past sentence summary 16P and the future sentence summary 16F related to the specific patient can be generated from the relation document 15 of the specific patient, and can be provided to the user.

Generation of Past Sentence Generation Mechanism Model 34 and Future Sentence Generation Mechanism Model 36

Next, a function of generating the past sentence generation mechanism model 34 and the future sentence generation mechanism model 36 in the medical care summary generation apparatus 10 according to the present embodiment will be described. Stated another way, a learning phase of the past sentence generation mechanism model 34 and the future sentence generation mechanism model 36 will be described.

FIG. 7 shows a functional block diagram of an example of a configuration related to the generation of the past sentence generation mechanism model 34 and the future sentence generation mechanism model 36 in the medical care summary generation apparatus 10 according to the present embodiment. As shown in FIG. 7, the medical care summary generation apparatus 10 comprises a past sentence and future sentence definition mechanism 60, a past sentence summary generation mechanism learning unit 64, and a future sentence summary generation mechanism learning unit 66. As an example, in the medical care summary generation apparatus 10 according to the present embodiment, in a case in which the CPU 20A of the controller 20 executes the learning program 32 stored in the storage 22C, the CPU 20A functions as the past sentence and future sentence definition mechanism 60, the past sentence summary generation mechanism learning unit 64, and the future sentence summary generation mechanism learning unit 66.

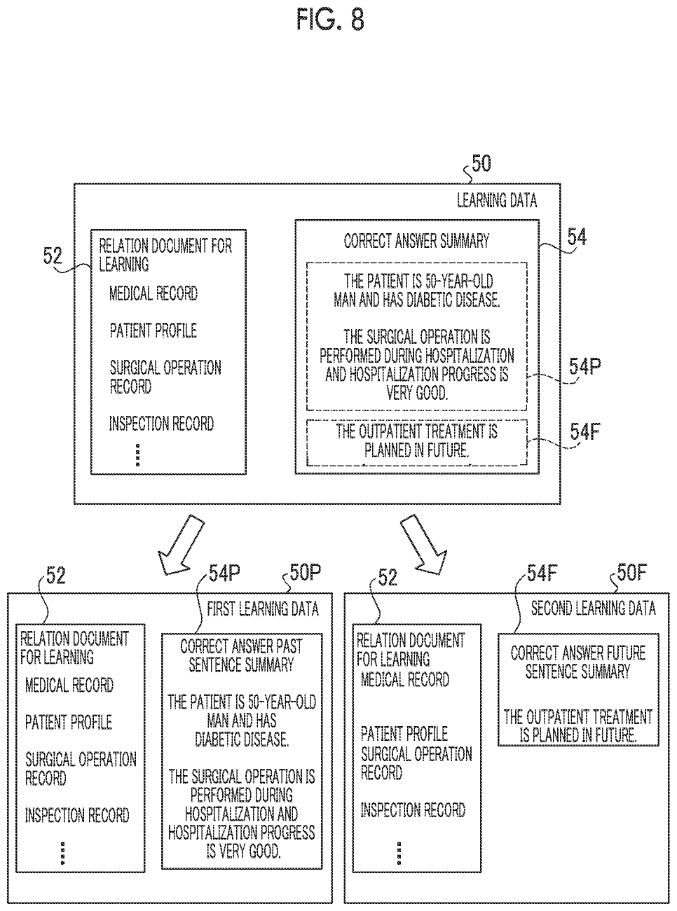

As shown in FIG. 8 as an example, in the medical care summary generation apparatus 10 according to the present embodiment, the learning data 50, which is a set of a relation document for learning 52 and a correct answer summary 54, is used to train the past sentence generation mechanism model 34 and the future sentence generation mechanism model 36. The learning data 50 is also called training data or teacher data. The relation document for learning 52 according to the present embodiment is an example of information data for learning according to the present disclosure, and the correct answer summary 54 according to the present embodiment is an example of document data for learning according to the present disclosure.

Similar to the relation document 15, the relation document for learning 52 includes the medical record of the specific patient, the patient profile, the surgical operation record, the inspection record, and the like. The correct answer summary 54 is a medical care summary actually generated by the doctor or the like with reference to the relation document for learning 52 related to the medical care of the specific patient. The correct answer summary 54 includes a correct answer past sentence summary 54P corresponding to the past sentence summary 16P, and a correct answer future sentence summary 54F corresponding to the future sentence summary 16F. The correct answer past sentence summary 54P according to the present embodiment is an example of a first portion according to the present disclosure, and the correct answer future sentence summary 54F according to the present embodiment is an example of a second portion according to the present disclosure.

In this regard, although in the present embodiment a past sentence is used as an example of the first portion and a future sentence is used as an example of the second portion, the disclosure is not limited thereto. For example, in a case in which the information data for leaning is limited to a portion of the patient information or patient data, such as to inspection data, a portion (or a description) of the patient information related to the inspection data may be used as the first portion and the other portion of the patient information may be used as the second portion. An example of the description in the patient information used as the second portion includes a description related to medical data other than the inspection data, a description related to diagnosis based on the inspection data (not the inspection data per se), and/or fixed phrases.

The past sentence and future sentence definition mechanism 60 extracts the correct answer past sentence summary 54P and the correct answer future sentence summary 54F having a lower rate of match with the relation document for learning 52 than the correct answer past sentence summary 54P from the correct answer summary 54 based on the rate of match between the relation document for learning 52 and each portion of the correct answer summary 54. As an example, the past sentence and future sentence definition mechanism 60 according to the present embodiment derives the rate of match with the relation document for learning 52 for each sentence included in the correct answer summary 54 by using an editing distance, which is a measure indicating a degree of difference (difference degree) between two character strings, recall-oriented understudy for gisting evaluation, which is an indicator for evaluating the summary, or the like. In addition, the past sentence and future sentence definition mechanism 60 uses a sentence in which the rate of match with the relation document for learning 52 is equal to or higher than a threshold value as the correct answer past sentence summary 54P. In other words, the past sentence and future sentence definition mechanism 60 uses a sentence in which the rate of match with the relation document for learning 52 is lower than the threshold value as the correct answer future sentence summary 54F.

For example, among the three sentences included in the correct answer summary 54 shown in FIG. 8, the rate of match of the sentence “The patient is a 50-year-old man and has a diabetic disease.” with the relation document for learning 52 is 70%, and the rate of match of the sentence “The surgical operation is performed during hospitalization and the hospitalization progress is very good.” with the relation document for learning 52 is 80%. In addition, among the three sentences included in the correct answer summary 54, the rate of match of the sentence “The outpatient treatment is planned in the future.” with the relation document for learning 52 is 10%. Here, in a case in which the threshold value as a criterion for determining whether or not there is a match is 20%, the past sentence and future sentence definition mechanism 60 extracts the former two sentences as the correct answer past sentence summary 54P and the latter one sentence as the correct answer future sentence summary 54F.

As a result, first learning data 50P, which is a set of the relation document for learning 52 and the correct answer past sentence summary 54P and is used to train the past sentence generation mechanism model 34, and second learning data 50F, which is a set of the relation document for learning 52 and the correct answer future sentence summary 54F and is used to train the future sentence generation mechanism model 36, are obtained.

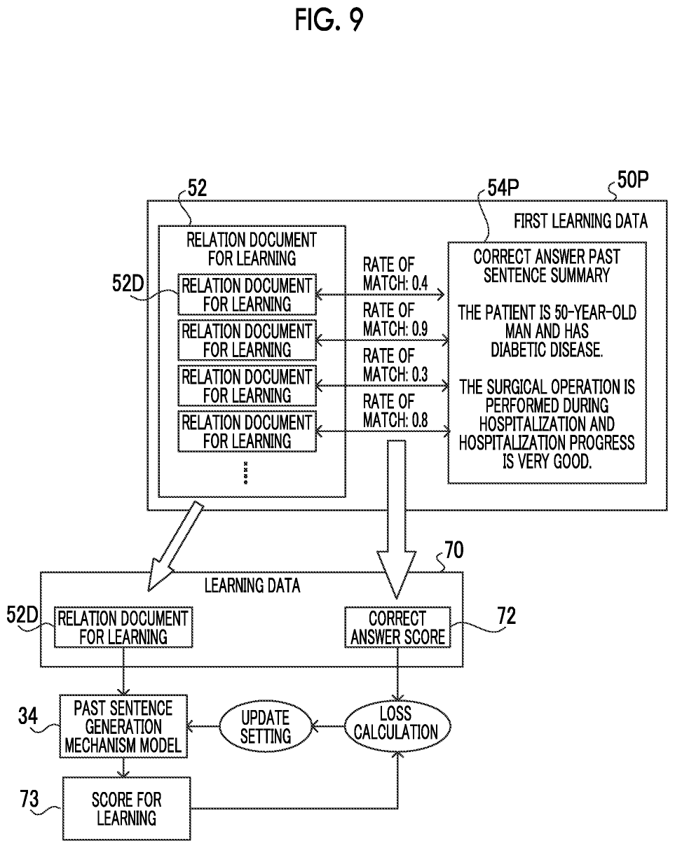

The past sentence summary generation mechanism learning unit 64 trains the machine learning model by using the first learning data 50P to generate the past sentence generation mechanism model 34 of the past sentence summary generation mechanism 44. FIG. 9 shows a diagram for describing training the past sentence generation mechanism model 34 by the past sentence summary generation mechanism learning unit 64 according to the present embodiment.

First, the past sentence summary generation mechanism learning unit 64 according to the present embodiment extracts one relation document for learning 52D from the relation document for learning 52 based on a predetermined criterion. As an example, the past sentence summary generation mechanism learning unit 64 according to the present embodiment extracts the relation document for learning 52D in units of a single sentence, by using one sentence included in the relation document for learning 52 as one relation document for learning 52D. It should be noted that a criterion for extracting the relation document for learning 52D from the relation document for learning 52 is not particularly limited, and for example, the criterion may be that the associated dates are the same day.

The past sentence summary generation mechanism learning unit 64 derives the rate of match of the relation document for learning 52D with the correct answer past sentence summary 54P. It should be noted that the past sentence summary generation mechanism learning unit 64 may adopt the highest rate of match among the rates of match with the sentences constituting the correct answer past sentence summary 54P as the rate of match with a certain relation document for learning 52D. For example, the correct answer past sentence summary 54P may be divided according to a predetermined condition, the rate of match with the relation document for learning 52D may be derived for each divided portion, and the highest rate of match of the portion may be used as the rate of match with the relation document for learning 52D. It should be noted that examples of the predetermined condition include a unit of a sentence, a unit of a phrase, and the like. In addition, examples of the method of dividing the correct answer past sentence summary 54P include a method of deriving the rate of match while using shifts for each character and dividing the correct answer past sentence summary 54P at a place in which the rate of match is highest. In addition, the method of deriving the rate of match by the past sentence summary generation mechanism learning unit 64 is not particularly limited. For example, the ROUGE described above or the like may be used. Alternately, for example, the rate of match may be manually set. The rate of match is derived for each individual relation document for learning 52D included in the relation document for learning 52. In the present embodiment, the rate of match is a value equal to or higher than 0 and equal to or lower than 1, and a higher numerical value indicates that the rate of match is higher, that is, there is a match. The past sentence generation mechanism model 34 is trained by being given learning data 70, which is a set of the relation document for learning 52D and a correct answer score 72 corresponding to this rate of match.

As shown in FIG. 9, in the learning phase, the past sentence generation mechanism model 34 according to the present embodiment is trained by being given the learning data 70, which is also called training data or teacher data.

In the learning phase, the relation document for learning 52D is vectorized for each word and input to the past sentence generation mechanism model 34, for example. The past sentence generation mechanism model 34 outputs a score for learning 73 for the relation document for learning 52D. Based on the correct answer score 72 and the score for learning 73, the loss calculation of the past sentence generation mechanism model 34 using a loss function is performed. Then, update settings of various coefficients of the past sentence generation mechanism model 34 are performed according to a result of the loss calculation, and the past sentence generation mechanism model 34 is updated according to the update settings.

It should be noted that, in this case, in the past sentence generation mechanism model 34, in addition to the relation document for learning 52D to be learned, additional information, such as an inspection value related to the patient, or the relation document for learning as another candidate may also be used as input. In this way, by using the relation information other than the relation document for learning 52D to be learned, it is possible to facilitate the prediction.

In the learning phase, the series of the processes described above of the input of the relation document for learning 52D to the past sentence generation mechanism model 34, the output of the score for learning 73 from the past sentence generation mechanism model 34, the loss calculation, the update setting, the update of the past sentence generation mechanism model 34 are repeated while exchanging the learning data 70. The repetition of the series of processes described above is terminated in a case in which prediction accuracy of the score for learning 73 with respect to the correct answer score 72 reaches a predetermined set level. In this way, the past sentence generation mechanism model 34 that uses a relation document 15D, which is the individual document included in the relation document 15, as input, and outputs the score is generated. A higher the score indicates that the input relation document 15D has a higher rate of match with the past sentence summary 16P to be generated. The past sentence summary generation mechanism learning unit 64 stores the generated past sentence generation mechanism model 34 in the storage 22C of the storage unit 22 of the medical care summary generation apparatus 10.

In this case, for example, in the operation phase, the past sentence summary generation mechanism 44 acquires the score for each relation document 15D output by inputting a plurality of relation documents 15D included in the relation document 15 to the past sentence generation mechanism model 34. Then, the past sentence summary generation mechanism 44 generates the past sentence summary 16P by extracting a predetermined number of the relation documents 15D from the plurality of relation documents 15D in descending order of the highest score.

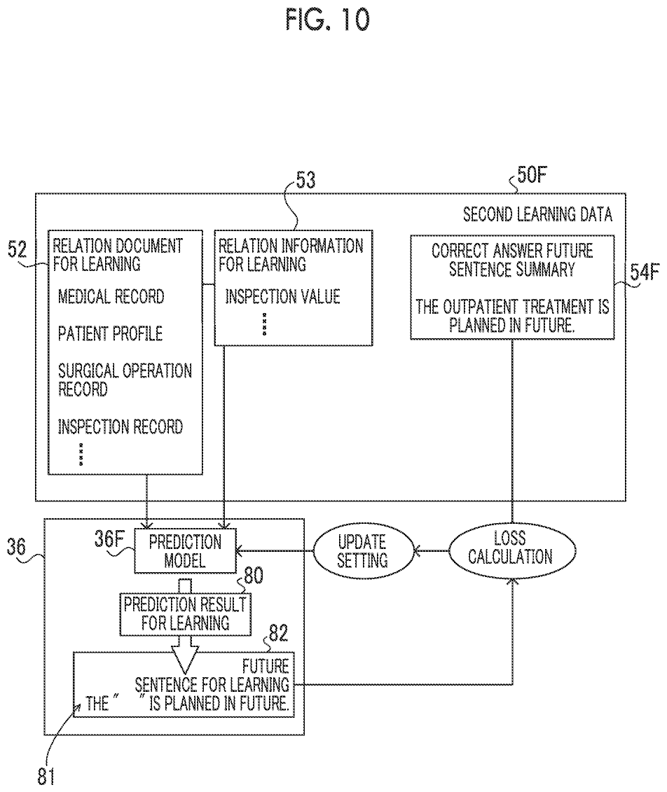

In addition, the future sentence summary generation mechanism learning unit 66 generates the future sentence generation mechanism model 36 of the future sentence summary generation mechanism 46 by training the machine learning model using the learning data 50 as input and using the correct answer future sentence summary 54F of the correct answer summary 54 as correct answer data. By training the machine learning model using the second learning data 50F, the future sentence generation mechanism model 36 of the future sentence summary generation mechanism 46 is generated. FIG. 10 shows a diagram for describing training the future sentence generation mechanism model 36 by the future sentence summary generation mechanism learning unit 66 according to the present embodiment.

As shown in FIG. 10, in the learning phase, the future sentence generation mechanism model 36 according to the present embodiment is trained by being given the second learning data 50F, which is also called training data or teacher data. The future sentence generation mechanism model 36 includes a prediction model 36F. The prediction model 36F is, for example, a prediction model that predicts a future state of the specific patient. In the learning phase, the relation document for learning 52 and relation information for learning 53, such as the inspection value associated with the specific patient, are input to the prediction model 36F. It should be noted that, for example, the document data, such as the relation document for learning 52, is vectorized for each document and input. The prediction model 36F outputs a prediction result for learning 80 to the relation document for learning 52. It should be noted that a task is adopted in which the aggregation of the correct answer future sentence summaries 54F and the template 81 are compared to extract higher-order substitution candidates, and the prediction result for learning 80 is selected from among higher-order substitution candidates. Examples of the template 81 used here include templates related to each of the outpatient treatment, the medication treatment, the home medical care, and the rehabilitation. In this case, training the prediction model 36F is not limited to this method, and a word to be substituted may be selected from a vocabulary set, a phrase set, a medical dictionary, or the like, or a criterion manually determined in advance (for example, the number of days until discharge from hospital (X to be substituted into “planned to be discharged from hospital after X days”)) may be set as the correct answer. It should be noted that, in a case in which the criterion manually determined in advance is used, it is assumed that learning data prepared separately (in this example, the number of days until discharge from hospital) is used to train the prediction model 36F. The future sentence generation mechanism model 36 generates the future sentence for learning 82 by combining the prediction result for learning 80 and the template 81. It should be noted that a form may be adopted in which the prediction result for learning 80 output by the prediction model 36F also includes contents, such as “outpatient treatment”, and information indicating the designation of the template 81 as to which template 81 to use. In this case, the future sentence generation mechanism model 36 combines the contents included in the prediction result for learning 80 with the designated template 81. The loss calculation of the prediction model 36F using the loss function is performed based on the future sentence for learning 82 and the correct answer future sentence summary 54F. Then, update settings of various coefficients of the prediction model 36F are performed according to a result of the loss calculation, and the prediction model 36F is updated according to the update settings.

In the learning phase, the series of the processes described above of the input of the relation document for learning 52 and the relation information for learning 53 to the prediction model 36F, the output of the prediction result for learning 80 from the prediction model 36F, the generation of the future sentence for learning 82 by combining the prediction result for learning 80 and the template 81, the loss calculation, the update setting, and the update of the prediction model 36F are repeatedly performed while exchanging the second learning data 50F. The repetition of the series of processes described above is terminated in a case in which prediction accuracy of the future sentence for learning 82 with respect to the correct answer future sentence summary 54F reaches a predetermined set level. The future sentence summary generation mechanism learning unit 66 stores the generated future sentence generation mechanism model 36 in the storage 22C of the storage unit 22 of the medical care summary generation apparatus 10.

It should be noted that the method of setting the template 81 is not particularly limited. For example, the template 81 may be manually set. Further, for example, a form may be adopted in which a plurality of types of the templates 81 are prepared, the learning is performed while changing the types of the templates 81, and the template 81 in which the future sentence for learning 82 most matches the correct answer future sentence summary 54F is adopted.

In addition, a form may be adopted in which the document is generated with the restriction of the prediction result for learning 80, instead of combining the prediction result for learning 80 and the template 81.

It should be noted that the method of generating the future sentence summary 54F is not limited to the form described above. For example, a form may be adopted in which the future sentence generation mechanism model 36 includes a single model for generating the future sentence. In this case, instead of the prediction model 36F, the single model is trained to output the future sentence for learning 82 by using the relation document for learning 52, or the second learning data 50F including the relation document for learning 52, the relation information for learning 53, and the correct answer future sentence summary 54F.

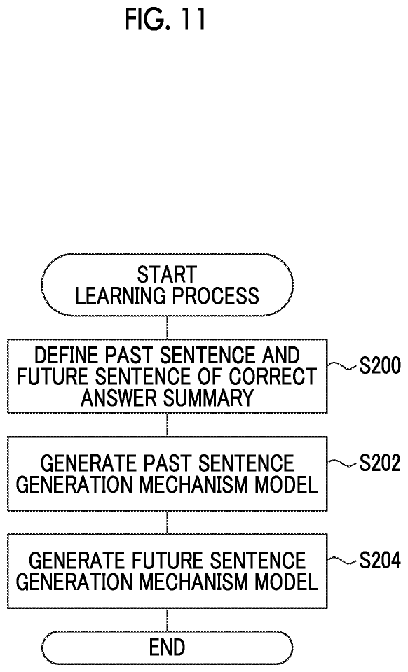

Next, an action of training the past sentence summary generation mechanism 44 and the future sentence summary generation mechanism 46 in the medical care summary generation apparatus 10 according to the present embodiment will be described with reference to the drawings. FIG. 11 shows a flowchart showing an example of a flow of a learning process executed in the medical care summary generation apparatus 10 according to the present embodiment. As an example, the medical care summary generation apparatus 10 according to the present embodiment executes the learning process shown in FIG. 11 as an example by the CPU 20A of the controller 20 executing the learning program 32 stored in the storage 22C based on, for example, a learning start instruction performed by the user using the operation unit 26.

In step S200, the past sentence and future sentence definition mechanism 60 defines the correct answer past sentence summary 54P and the correct answer future sentence summary 54F in the correct answer summary 54 of the learning data 50. As described above, the past sentence and future sentence definition mechanism 60 according to the present embodiment extracts the correct answer past sentence summary 54P and the correct answer future sentence summary 54F having a lower rate of match with the relation document for learning 52 than the correct answer past sentence summary 54P from the correct answer summary 54 based on the rate of match between the relation document for learning 52 and each portion of the correct answer summary 54.

In next step S202, the past sentence summary generation mechanism learning unit 64 generates the past sentence generation mechanism model 34 by using the first learning data 50P. As described above, the past sentence summary generation mechanism learning unit 64 according to the present embodiment derives the rate of match between each of the relation document for learning 52D included in the relation document for learning 52 of the first learning data 50P and the correct answer past sentence summary 54P. In addition, the past sentence summary generation mechanism learning unit 64 derives the correct answer score 72 of each relation document for learning 52D based on the derived rate of match, and trains the past sentence generation mechanism model 34 by using the learning data 70 including the relation document for learning 52D and the correct answer score 72.

In next step S204, the future sentence summary generation mechanism learning unit 66 generates the future sentence generation mechanism model 36 by using the second learning data 50F. As described above, the future sentence summary generation mechanism learning unit 66 according to the present embodiment inputs the relation document for learning 52 to the prediction model 36F. The future sentence generation mechanism model 36 is generated by updating the future sentence generation mechanism model 36 using the future sentence for learning 82 that combines the prediction result for learning 80 output from the prediction model 36F and the template 81, and the correct answer future sentence summary 54F. In a case in which the process of step S204 is terminated, the learning process shown in FIG. 11 is terminated.

By training the past sentence generation mechanism model 34 and the future sentence generation mechanism model 36 in this way, the medical care summary generation apparatus 10 can generate the medical care summary 16 from the relation document 15, as described above.

It should be noted that the past sentence and future sentence definition mechanism 60 need only to be able to extract the correct answer past sentence summary 54P and the correct answer future sentence summary 54F from the correct answer summary 54. The method of the extraction or the like is not limited to the method described above, and for example, a modification example 1 may be adopted.

### Modification Example 1

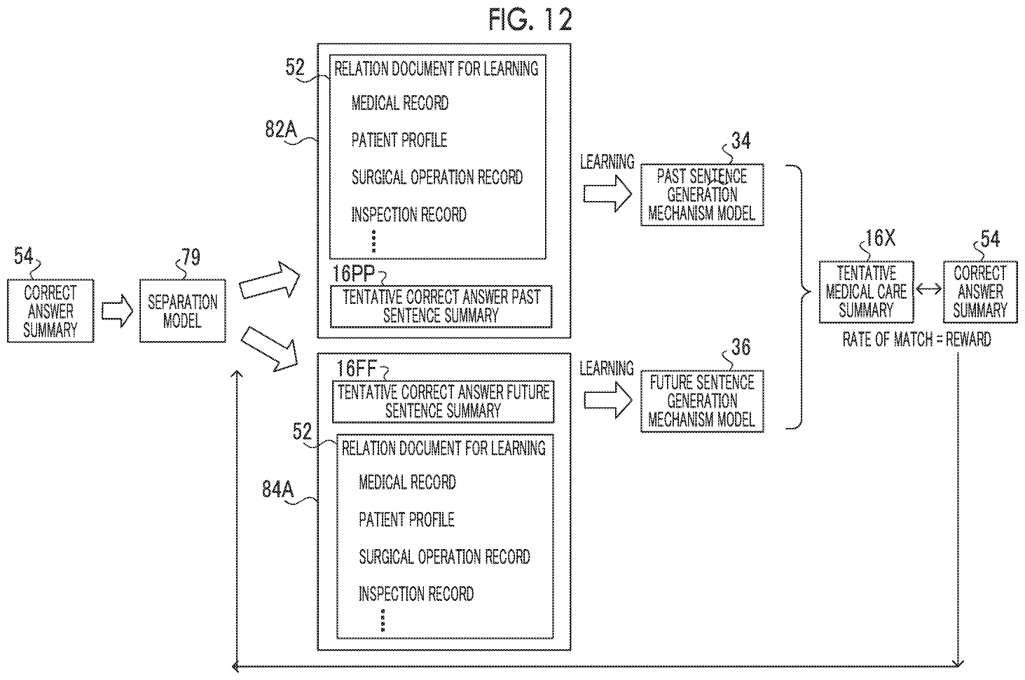

FIG. 12 shows a diagram for describing the extraction of the correct answer past sentence summary 54P and the correct answer future sentence summary 54F by the past sentence and future sentence definition mechanism 60 according to the present modification example. In the present modification example, the correct answer summary 54 is separated into the correct answer past sentence summary 54P and the correct answer future sentence summary 54F by a separation model 79. In the learning phase of the separation model 79, as shown in FIG. 12, the separation model 79 uses the correct answer summary 54 as input and separates a tentative correct answer past sentence summary 16PP and a tentative correct answer future sentence summary 16FF. The separation model 79 separates a plurality of correct answer summaries 54 to generate a plurality of tentative correct answer past sentence summaries 16PP and a plurality of tentative correct answer future sentence summaries 16FF. Further, the past sentence generation mechanism model 34 is trained by using learning data 82A in which the relation document for learning 52 and the plurality of tentative correct answer past sentence summaries 16PP are combined, and a tentative past sentence summary is generated by the trained past sentence generation mechanism model 34. In addition, the future sentence generation mechanism model 36 is trained by learning data 84A in which the relation document for learning 52 and the plurality of tentative correct answer future sentence summaries 16FF are combined, and a tentative future sentence summary is generated by the trained future sentence generation mechanism model 36. Further, a tentative medical care summary 16X is generated by combining the tentative past sentence summary and the tentative future sentence summary. By performing the reinforcement learning of the separation model 79 with the rate of match between the tentative medical care summary 16X and the correct answer summary 54 as a reward, the separation model 79 can appropriately extract the correct answer past sentence summary 54P and the correct answer future sentence summary 54F from the correct answer summary 54. It should be noted that the separation model 79 may be a model that outputs one of the correct answer past sentence summary 54P or the correct answer future sentence summary 54F. The separation model 79 according to the present modification example is an example of a third machine learning model according to the present disclosure.

In addition, the past sentence summary generation mechanism learning unit 64 need only to be able to generate the past sentence generation mechanism model 34, and the method of the generation, the specific contents of the generated past sentence generation mechanism model 34, or the like is not limited to the form described above. For example, the past sentence summary generation mechanism learning unit 64 may further train a machine learning model that rearranges the scores of the relation documents 15D extracted as described above in descending order of the highest score.

In addition, the future sentence summary generation mechanism learning unit 66 need only to be able to generate the future sentence generation mechanism model 36, and the method of the generation, the specific contents of the generated future sentence generation mechanism model 36, or the like is not limited to the form described above. For example, a form may be adopted in which the relation document 15D closest to the future sentence summary 16F is extracted from the relation document 15D included in the patient information, and the extracted relation document 15D is rewritten into the future sentence. In addition, a form may be adopted in which a part of the prediction model 36F and a part of the machine learning model that generates the template 81 may be provided as a common model, and the future sentence generation mechanism model 36 may comprise three models.

As described above, in the medical care summary generation apparatus 10 according to the embodiment described above, the CPU 20A acquires the learning data 50 including the relation document for learning 52 and the correct answer summary 54.

Based on the rate of match between the relation document for learning 52 and each portion of the relation document for learning 52, the correct answer past sentence summary 54P and the correct answer future sentence summary 54F having a higher rate of match with the relation document for learning 52 than the correct answer past sentence summary 54P are extracted from the correct answer summary 54. The past sentence generation mechanism model 34 is generated from the first learning data in which the relation document for learning 52 is used as the input data and the correct answer past sentence summary 54P is used as the correct answer data. The future sentence generation mechanism model 36 is generated from the second learning data in which the relation document for learning 52 included in the relation document for learning 52 is used as the input data and the correct answer future sentence summary 54F is used as the correct answer data.

As described above, the medical care summary generation apparatus 10 according to the present embodiment generates the past sentence generation mechanism model 34 by using the correct answer past sentence summary 54P, which has a high rate of match with the relation document for learning 52 and is relatively easy to directly derive, as the correct answer data. In addition, the medical care summary generation apparatus 10 generates the future sentence generation mechanism model 36 by using the correct answer future sentence summary 54F, which has a low rate of match with the relation document for learning 52 and is relatively difficult to directly derive, as the correct answer data.

As a result, the medical care summary generation apparatus 10 according to the present embodiment generates the past sentence summary 16P from the relation document 15 by using the past sentence generation mechanism model 34, and generates the future sentence summary 16F by using the future sentence generation mechanism model 36. Moreover, the medical care summary generation apparatus 10 can generate the medical care summary 16 from the past sentence summary 16P and the future sentence summary 16F. The past sentence summary 16P having a high degree of association to the relation document 15 and the future sentence summary 16F having a low degree of association to the relation document 15 can be appropriately generated from the relation document 15. Therefore, with the medical care summary generation apparatus 10 according to the present embodiment, it is possible to generate an appropriate medical care summary 16.

It should be noted that, in the form described above, the form is described in which the medical care summary 16 including the past sentence summary 16P and the future sentence summary 16F is generated from the patient information related to the specific patient, but the target to be generated is not limited to the present form. For example, the technology of the present disclosure may be applied to a form in which a market report including the past sentence summary 16P and the future sentence summary 16F is generated from purchaser information including a purchaser profile related to a specific purchaser, a history of a purchased item, purchase date and time, and the like.

Moreover, in the embodiment described above, for example, various processors described below can be used as the hardware structure of processing units that execute various processes, such as the medical care summary generation unit 40, the past sentence summary generation mechanism 44, the future sentence summary generation mechanism 46, the display controller 48, the past sentence and future sentence definition mechanism 60, the past sentence summary generation mechanism learning unit 64, and the future sentence summary generation mechanism learning unit 66. As described above, the various processors include, in addition to the CPU that is a general-purpose processor that executes software (program) to function as various processing units, a programmable logic device (PLD) that is a processor of which a circuit configuration can be changed after manufacture, such as a field programmable gate array (FPGA), and a dedicated electric circuit that is a processor having a circuit configuration that is designed for exclusive use in order to execute a specific process, such as an application specific integrated circuit (ASIC).

One processing unit may be configured by using one of the various processors or may be configured by using a combination of two or more processors of the same type or different types (for example, a combination of a plurality of FPGAs or a combination of a CPU and an FPGA). In addition, a plurality of the processing units may be configured by using one processor.

A first example of the configuration in which the plurality of processing units are configured by using one processor is a form in which one processor is configured by using a combination of one or more CPUs and the software and this processor functions as the plurality of processing units, as represented by computers, such as a client and a server. A second example thereof is a form of using a processor that realizes the function of the entire system including the plurality of processing units by one integrated circuit (IC) chip, as represented by a system on chip (SoC) or the like. In this way, as the hardware structure, the various processing units are configured by using one or more of the various processors described above.

Further, more specifically, as the hardware structure of the various processors, an electric circuit (circuitry) in which circuit elements, such as semiconductor elements, are combined can be used.

In addition, in each embodiment described above, the aspect is described in which each of the medical care summary generation program 30 and the learning program 32 is stored (installed) in the storage unit 22 in advance, but the present disclosure is not limited to this. Each of the medical care summary generation program 30 and the learning program 32 may be provided in a form being recorded on a recording medium, such as a compact disc read only memory (CD-ROM), a digital versatile disc read only memory (DVD-ROM), and a universal serial bus (USB) memory. In addition, a form may be adopted in which each of the medical care summary generation program 30 and the learning program 32 is downloaded from an external apparatus via a network. That is, a form may be adopted in which the program described in the present embodiment (program product) is distributed from an external computer, in addition to the provision by the recording medium.

In regard to the embodiment described above, the following additional notes will be further disclosed.

Additional Note 1

A model generation apparatus comprising at least one processor, in which the processor acquires information data for learning, acquires document data for learning, extracts a first portion and a second portion having a lower rate of match with the information data for learning than the first portion from the document data for learning based on a rate of match between the information data for learning and each portion of the document data for learning, generates a first machine learning model by using first learning data in which first data for learning included in the information data for learning is used as input data and the first portion is used as correct answer data, and generates a second machine learning model by using second learning data in which second data for learning included in the information data for learning is used as input data and the second portion is used as correct answer data.

Additional Note 2

The model generation apparatus according to additional note 1, in which the document data for learning is patient data related to a specific patient with which first date information is associated, and the information data for learning includes a plurality of document data which are patient data related to the specific patient with which the first date information or second date information indicating a date earlier than a date indicated by the first date information is associated.

Additional Note 3

The model generation apparatus according to additional note 1 or 2, in which the processor generates a third machine learning model that uses the document data for learning as input, and outputs at least one of the first portion or the second portion through reinforcement learning in which performance of the first machine learning model and performance of the second machine learning model are used as rewards, and extracts the first portion and the second portion from the document data for learning by using the third machine learning model.

Additional Note 4

The model generation apparatus according to any one of additional notes 1 to 3, in which the second machine learning model is a machine learning model that includes a machine learning model outputting a prediction result based on the information data for learning, and outputs a combination of the prediction result and a template.

Additional Note 5

A document generation apparatus comprising a first machine learning model generated by using first learning data in which first data for learning included in information data for learning is used as input data and a first portion extracted from document data for learning based on a rate of match between the information data for learning and each portion of the document data for learning is used as correct answer data, a second machine learning model generated by using second learning data in which second data for learning included in the information data for learning is used as input data and a second portion, which is extracted from the document data for learning and has a lower rate of match with the information data for learning than the first portion, is used as correct answer data, and at least one processor, in which the processor acquires information data, acquires a first document by inputting first data included in the information data to the first machine learning model, acquires a second document by inputting second data included in the information data to the second machine learning model, and generates a third document from the first document and the second document.

Additional Note 6

A model generation method executed by a processor of a model generation apparatus including at least one processor, the model generation method comprising acquiring information data for learning, acquiring document data for learning, extracting a first portion and a second portion having a lower rate of match with the information data for learning than the first portion from the document data for learning based on a rate of match between the information data for learning and each portion of the document data for learning, generating a first machine learning model by using first learning data in which first data for learning included in the information data for learning is used as input data and the first portion is used as correct answer data, and generating a second machine learning model by using second learning data in which second data for learning included in the information data for learning is used as input data and the second portion is used as correct answer data.

Additional Note 7

A document generation method executed by a processor of a document generation apparatus including a first machine learning model generated by using first learning data in which first data for learning included in information data for learning is used as input data and a first portion extracted from document data for learning based on a rate of match between the information data for learning and each portion of the document data for learning is used as correct answer data, a second machine learning model generated by using second learning data in which second data for learning included in the information data for learning is used as input data and a second portion, which is extracted from the document data for learning and has a lower rate of match with the information data for learning than the first portion, is used as correct answer data, and at least one processor, the document generation method comprising acquiring information data, acquiring a first document by inputting first data included in the information data to the first machine learning model, acquiring a second document by inputting second data included in the information data to the second machine learning model, and generating a third document from the first document and the second document.

Additional Note 8

A program for executing at least one of the model generation method according to additional note 6 or the document generation method according to additional note 7.

What is claimed is:

1. A model generation apparatus comprising:
at least one processor that is configured to:
acquire information data for learning,
acquire document data for learning,
extract a first portion and a second portion having a lower rate of match with the information data for learning than the first portion from the document data for learning based on a rate of match between the information data for learning and each portion of the document data for learning,
generate a first machine learning model by using first learning data in which first data for learning included in the information data for learning is used as input data and the first portion is used as correct answer data, and
generate a second machine learning model by using second learning data in which second data for learning included in the information data for learning is used as input data and the second portion is used as correct answer data.

2. The model generation apparatus according to claim 1,
wherein the document data for learning is patient data related to a specific patient with which first date information is associated, and
the information data for learning includes a plurality pieces of document data which are patient data related to the specific patient with which the first date information or second date information indicating a date earlier than a date indicated by the first date information is associated.

3. The model generation apparatus according to claim 1,
wherein the at least one processor is configured to:
generate a third machine learning model that uses the document data for learning as input, and outputs at least one of the first portion or the second portion through reinforcement learning in which performance of the first machine learning model and performance of the second machine learning model are used as rewards, and
extract the first portion and the second portion from the document data for learning by using the third machine learning model.

4. The model generation apparatus according to claim 1,
wherein the second machine learning model is a machine learning model that includes a machine learning model outputting a prediction result based on the information data for learning, and outputs a combination of the prediction result and a template.

5. A document generation apparatus comprising:
a first machine learning model generated by using first learning data in which first data for learning included in information data for learning is used as input data and a first portion extracted from document data for learning based on a rate of match between the information data for learning and each portion of the document data for learning is used as correct answer data;
a second machine learning model generated by using second learning data in which second data for learning included in the information data for learning is used as input data and a second portion, which is extracted from the document data for learning and has a lower rate of match with the information data for learning than the first portion, is used as correct answer data; and
at least one processor that is configured to:
acquire information data,
acquire a first document by inputting first data included in the information data to the first machine learning model,
acquire a second document by inputting second data included in the information data to the second machine learning model, and
generate a third document from the first document and the second document.

6. A model generation method executed by at least one processor of a model generation apparatus including the at least one processor, the model generation method comprising:
acquiring information data for learning;
acquiring document data for learning;
extracting a first portion and a second portion having a lower rate of match with the information data for learning than the first portion from the document data for learning based on a rate of match between the information data for learning and each portion of the document data for learning;
generating a first machine learning model by using first learning data in which first data for learning included in the information data for learning is used as input data and the first portion is used as correct answer data; and
generating a second machine learning model by using second learning data in which second data for learning included in the information data for learning is used as input data and the second portion is used as correct answer data.

7. A document generation method executed by at least one processor of a document generation apparatus, the document generation apparatus including:
a first machine learning model generated by using first learning data in which first data for learning included in information data for learning is used as input data and a first portion extracted from document data for learning based on a rate of match between the information data for learning and each portion of the document data for learning is used as correct answer data,
a second machine learning model generated by using second learning data in which second data for learning included in the information data for learning is used as input data and a second portion, which is extracted from the document data for learning and has a lower rate of match with the information data for learning than the first portion, is used as correct answer data, and
at least one processor,
the document generation method comprising:
acquiring information data;
acquiring a first document by inputting first data included in the information data to the first machine learning model;
acquiring a second document by inputting second data included in the information data to the second machine learning model; and
generating a third document from the first document and the second document.

8. A non-transitory storage medium storing a program causes a computer to execute a model generation processing, the model generation processing comprising:
acquiring information data for learning;
acquiring document data for learning;
extracting a first portion and a second portion having a lower rate of match with the information data for learning than the first portion from the document data for learning based on a rate of match between the information data for learning and each portion of the document data for learning;
generating a first machine learning model by using first learning data in which first data for learning included in the information data for learning is used as input data and the first portion is used as correct answer data; and
generating a second machine learning model by using second learning data in which second data for learning included in the information data for learning is used as input data and the second portion is used as correct answer data.

9. A non-transitory storage medium storing a program causes a computer to execute a document generation processing, the model generation processing comprising:
preparing a first machine learning model generated by using first learning data in which first data for learning included in information data for learning is used as input data and a first portion extracted from document data for learning based on a rate of match between the information data for learning and each portion of the document data for learning is used as correct answer data;
preparing a second machine learning model generated by using second learning data in which second data for learning included in the information data for learning is used as input data and a second portion, which is extracted from the document data for learning and has a lower rate of match with the information data for learning than the first portion, is used as correct answer data;
acquiring information data;
acquiring a first document by inputting first data included in the information data to the first machine learning model;
acquiring a second document by inputting second data included in the information data to the second machine learning model; and
generating a third document from the first document and the second document.
